(19) United States
(12) Patent Application Publication
(10) Pub. No.: US 2008/0216217 A1
(43) Pub. Date: Sep. 11, 2008

(54) Snow goggles

(76) Inventor: Kuei-Hsueh Wang, Tainan Hsien (TW)

Correspondence Address: Kuei-Hsueh Wang, P.O. Box 6-9 (717), Tainan, TW

(21) Appl. No.: 11/715,724

(22) Filed: Mar. 9, 2007

(51) Int. Cl. A61F 9/02

(52) U.S. Cl. 2/426

(57) ABSTRACT

A pair of snow goggles includes a frame, two lenses, a band and a light unit. The light unit consists of a base, a plurality of lamps, a battery, and a changeover switch. The frame is provided with air holes for air to flow in and out of the frame, and a plurality of round holes for the lamps to protruding out, and a fitting groove for the light unit to fit tightly therein. Thus, the lamps can be lit up for showing an emergency to attract attention of rescuers by a skier wearing this snow goggles in case of getting lost or coming across an accident.

### BACKGROUND OF THE INVENTION

1. Field of the Invention

This invention relates to a snow goggles, particularly to one provided with a light unit usable for indicating an emergency signal by skiers.

2. Description of the Prior Art

A conventional snow goggles are not provided with a light unit, as shown in FIG. 1, so provided a skier should come across a dangerous situation, or an accident, and needs an emergency rescue, the person might lose a precious moment for calling attention for someone to come to rescue owing to no means of showing any signal of letting others know the person's location.

### SUMMARY OF THE INVENTION

This invention has been devised to offer a snow goggles additionally provided with a light unit for to be lit up to show an emergency or accident by a skier in case getting lost or coming across an accident.

The feature of the invention is a light unit fitted in a fitting groove of a frame of the snow goggles, and the light unit is composed of a base, a plurality of lamps preferably formed with various colored LED, a battery, and a changeover switch electrically connected with one another.

### BRIEF DESCRIPTION OF DRAWINGS

This invention will be better understood by referring to the accompanying drawings, wherein:

FIG. 1 is a perspective view of a pair of conventional snow goggles;

FIG. 2 is an exploded perspective view of a pair of snow goggles in the present invention;

FIG. 3 is a partial perspective view of the snow goggles in the present invention;

FIG. 4 is a perspective view of the snow goggles worn on a person's head in the present invention; and,

FIG. 5 is a perspective view of the snow goggles used by a rider of a motorcycle or a bicycle in the present invention.

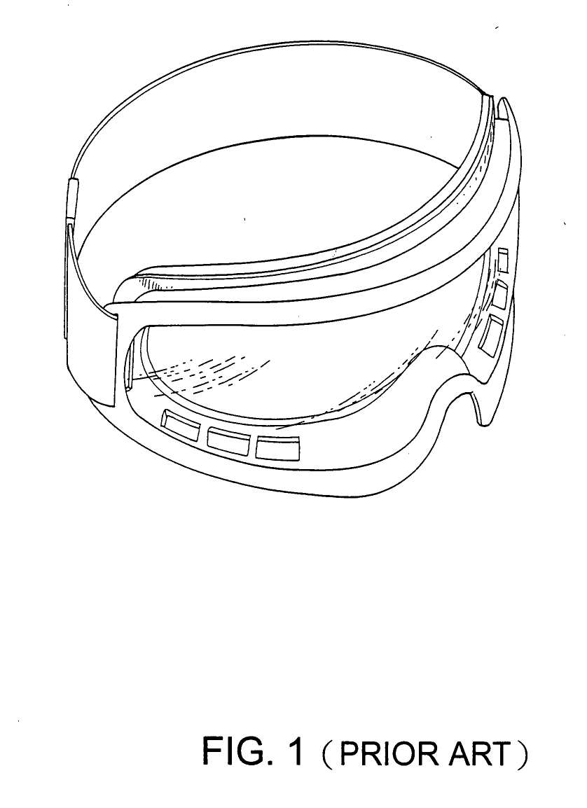

### DETAILED DESCRIPTION OF THE PREFERRED EMBODIMENT

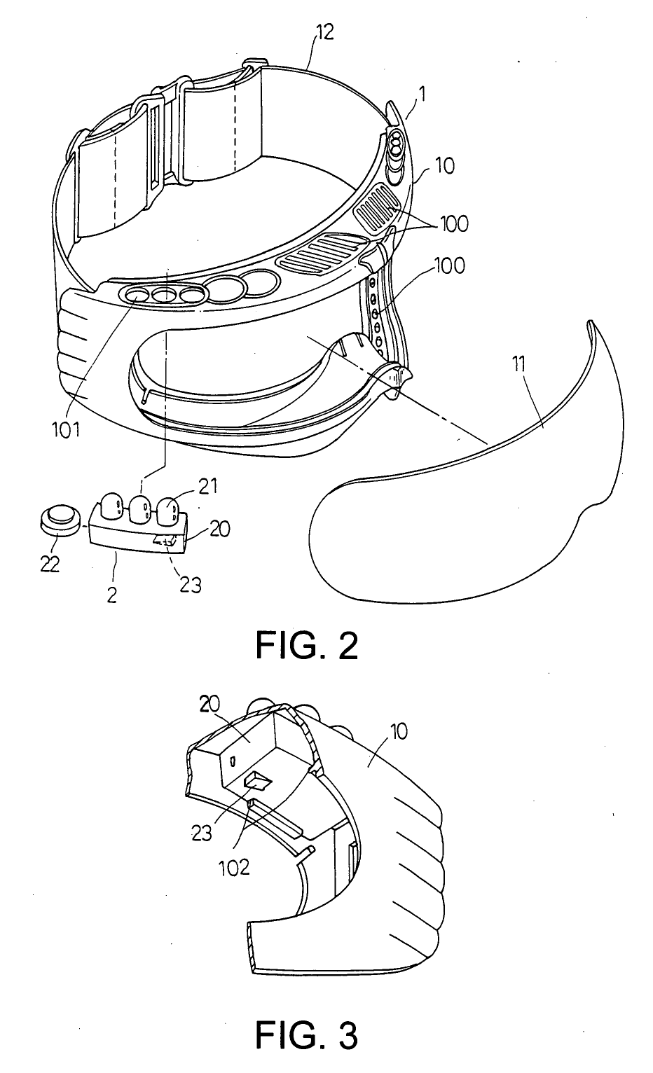

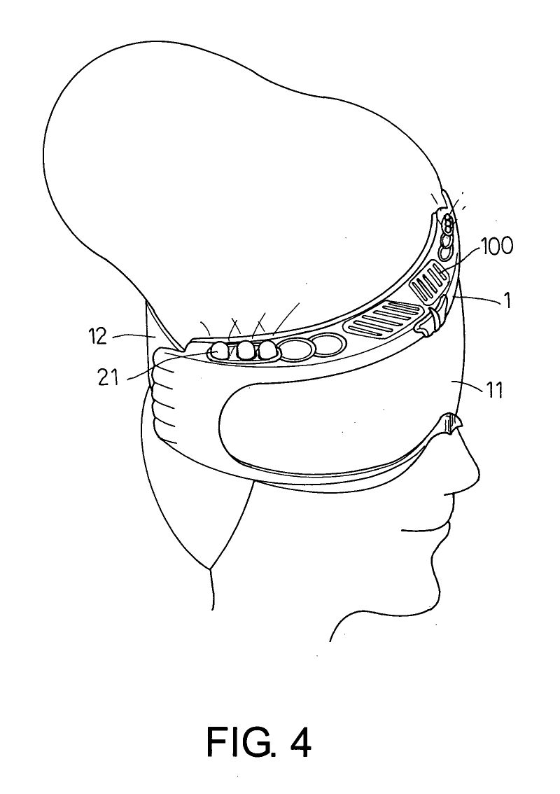

A preferred embodiment of a pair of snow goggles 1 in the present invention, as shown in FIGS. 2, 3 and 4, includes a frame 10, two lenses 1.1, a band 12, and a light unit 2, as main components.

The frame 10 is provided with a plurality of air holes 100 in an upper and lower end portions, round holes 101 and a fitting groove 102.

The light unit 2 is fitted in the fitting groove 102, consisting of a base 20, a plurality of lamps 21, a battery 22, and a changeover switch 23. The lamps 21, the battery 22 and the changeover switch 23 are fixed on the base 20, the lamps 21 are preferably LED (light emitting diodes) with various colors or bulb lamps, and the changeover switch 23 is connected with an IC circuit board, electrically connected with the lamps 21 and the battery 22, forming a circuit.

In assembling and using, referring to FIG. 2, firstly, the light unit 2 is positioned in the fitting groove 102 of the frame 10, and the lamps 21 protrude through out of the round holes 101 to be secured stably. While A skier wearing this snow goggles is skiing, the skier turns on the changeover switch 23 of the light unit 2, lighting up the lamps 21 in case of getting lost or an accident, with no needs of carrying a flashlight in one hand. Then, a rescuer can easily notice the lit up lamps of the snow goggles 1 so the rescuer can quickly make his way to the location where the skier with the goggles is.

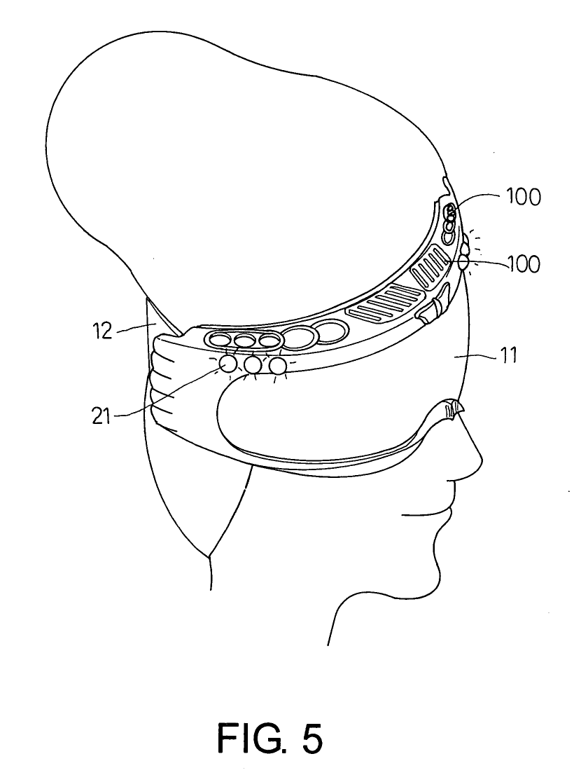

This invention has the following advantages, as can be seen from the above description.
1. A skier wearing this snow goggles 1 needs no other appliance, with the light unit 2 increasing its practicability.
2. The light unit 2 of the snow goggles 1 can serve a user in case of emergency as an emergency light by lighting up the lamps 21 for attracting rescuer's attention to let them take notice at once the location of a skier trapped in an accident.
3. The air holes 100 provided in an upper and a lower end portion of the frame 10 can permit air to ventilate in and out for the heat of the head of a wearer to disperse out so the skier may feel comfortable.
4. The lamp unit 2 can be fixed to face forward as shown in FIG. 5, and riders of motorcycles or bicycles can wear to let the lamps lit up to show a vivid mark for lowering accidents.
5. The lamps 21 can be made to flash on-and-off by the IC circuit.
6. The light unit 2 is fitted in the frame 10, not affecting the beauty of the whole goggles.

While the preferred embodiment of the invention has been described above, it will be recognized and understood that various modifications may be made therein and the appended claims are intended to cover all such modifications that may fall within the spirit and scope of the invention.

What is claimed is:

1. A pair of snow goggles comprising:
a frame provided with a plurality of air holes in an upper and a lower end portion, plural round holes for lamps to protruding out, and a fitting groove for a light unit to fit therein and secured stably;
a light unit is composed of a base, a plurality of lamps formed of various colored LED, a battery supplying electricity to said lamps, and a changeover switch connected with an IC circuit for turning on and off said lamps, said lamps, said battery and said changeover switch all arranged on said base.
two lenses fitted in the frame for a user to look through; and,
a band connected with said frame for a user to wear said snow goggles on the head.
